(19) United States
(12) Patent Application Publication
(10) Pub. No.: US 2006/0121499 A1
(43) Pub. Date: Jun. 8, 2006

(54) Methods of identifying glucocorticoids without the detrimental side effects of bone loss

(76) Inventors: Stavros C. Manolagas, Little Rock, AR (US); Teresita Bellido, Little Rock, AR (US)

Correspondence Address: McTavish Patent Firm, 429 Birchwood Courts, Birchwood, MN, 55110, US

(21) Appl. No.: 11/236,042

(22) Filed: Sep. 27, 2005

Related U.S. Application Data
(60) Provisional application No. 60/614,159, filed on Sep. 28, 2004.

(51) Int. Cl. C12Q 1/68

(52) U.S. Cl. 435/6

(57) ABSTRACT

The invention pertains to the elucidation of the pro-apoptotic effect of glucocorticoids on osteocytes. The present invention provides methods to screen compounds that retain the anti-inflammatory properties of glucocorticoids yet do not result in the detrimental effect of bone loss and compound identified by the methods.

### RELATED APPLICATION

This application claims the benefit of U.S. Provisional Application No. 60/614,159, filed Sep. 28, 2004. The entire teachings of the above application is incorporated herein by reference.

### GOVERNMENT SUPPORT

The invention was supported, in whole or in part, by grants K02-AR02127 and P01-AG13918 from National Institute of Health and a VA Merit Review and Research Enhancement Award Project from the Department of Veterans Affairs. The Government has certain rights in the invention.

### BACKGROUND OF THE INVENTION

Glucocorticoids are powerful anti-inflammatory compounds that have the ability to inhibit all stages of the inflammatory response. Common glucocorticoids include prednisone, dexamethasone and hydrocortisone. While glucocorticoids are widely used as drugs to treat various inflammatory conditions, prolonged use may have adverse side effects such as immunosuppression, fluid shifts, brain changes, and psychological changes. Further glucocorticoids have been found to increase blood glucose levels as well as suppress calcium absorption through their various metabolic effects. More severe side effects such as diabetes or osteoporosis can also occur.

The adverse effects of hypercortisolism on bone have been recognized for over 70 years (Cushing, H., Bull. Johns Hopkins Hosp., 50:137-195 (1932)), but the precise cellular and molecular basis of these changes has remained elusive. Today, the iatrogenic form of the disease has become far more common than Cushing's syndrome and glucocorticoid-induced osteoporosis is now third in frequency following post-menopausal and senile osteoporosis (Lukert, B., Glucocorticoid-induced osteoporosis. In Osteoposis, R. Marcus., et al. Eds., Academic Press, San Diego, Calif. 801-820 (1996)).

Bone loss due to glucocorticoid excess is diffuse, affecting both cortical and cancellous bone, but has a predilection for the axial skeleton. Spontaneous fractures of the vertebrae or ribs are, therefore, often presenting manifestations of the disorder (Fitzpatrick, L. A. Glucocorticoid-induced osteoporosis. In Osteoporosis. R. Marcus, editor. Blackwell Scientific Publications, Boston, Mass. 202-226 (1994) and Reid, I. R. Clin. Endocrinol. 30:83-103(1989)).

A cardinal feature of glucocorticoid-induced osteoporosis is decreased bone formation (Dempster, D. J., Bone Miner. Res. 4:137-141(1989)). In addition, patients receiving long-term glucocorticoid therapy sometimes develop collapse of the femoral head (osteonecrosis), but the mechanism underlying this is uncertain (Mankin, H. J., N. Engl. J. Med., 326:1473-1479 (1992)). Decreased bone formation, and in situ death of isolated segments of the proximal femur suggest that glucocorticoid excess may alter the birth and death of bone cells (Weinstein R. S., et al., J. Clin. Invest.,102(2):274-282(1998); O'Brien, C. A. et al., Endocrinology, 145(4):1835-41(2004) and Weinstein R. S. et al., Endocrinology,145(4):1980-7 (2004)).

Defective osteoblastogenesis has been reported to be linked to reduced bone formation and age-related osteopenia in the SAMP6 mouse (Jilka, R. L., et al. J. Clin. Invest. 97:1732-1740(1996)). Besides the relationship between aberrant osteoblast production and osteoporosis, it has been shown that a significant proportion of osteoblasts undergo apoptosis (Jilka, R. L., et al. J. Bone Miner. Res. 13 (1998), which raises the possibility that the premature or more frequent occurrence of osteoblast apoptosis could contribute to incomplete repair of resorption cavities and loss of bone.

Thus, a need exists for identifying compounds that possess the advantageous properties of glucocorticoids, in particular, anti-inflammatory properties, but do not cause detrimental side effects, such as bone loss or osteoporosis.

### SUMMARY OF THE INVENTION

It has now been found that activation of the glucocorticoid receptor mediated nontranscriptional pathway causes the bone deteriorating effects of glucocorticoids. Based on this discovery, methods of screening for anti-inflammatory compounds without these undesired side effects, anti-inflammatory compounds without these side effects and methods of using the same are disclosed herein.

In one embodiment, a method of screening for an anti-inflammatory compound that has reduced side effects, for example, the bone deteriorating side effects of glucocorticoid is described. The ability of the compound to activate the genomic activity mediated by the receptor is assessed and the ability of the compound to activate the nongenomic activity mediated by the receptor is assessed. If the compound activates the genomic activity mediated by the receptor without substantially activating the nongenomic activity mediated by the receptor, the compound is assayed for anti-inflammatory activity.

In certain embodiments, methods are disclosed for screening for an anti-inflammatory compound, comprising: contacting a cell containing a glucocorticoid receptor with a test compound; determining whether a test compound activates the genomic activity mediated by the receptor; determining the level of nongenomic activity mediated by the receptor. If the compound activates the genomic activity mediated by the receptor without substantially activating the nongenomic activity mediated by the receptor, the compound is then assayed for anti-inflammatory activity. As recited herein, “without substantially activating” means the activation is no greater than about 10% compared to basal levels, in certain embodiment, the activation is no greater than about 20% compared to basal levels, and in other embodiments, the activation is no greater than 50% compared to basal levels. In a further aspect of this embodiment, if the compound activates the genomic activity mediated by the receptor without substantially activating the nongenomic activity by the receptor and has anti-inflammatory activity; the compound is further assayed to determine the level of bone deteriorating activity.

In another embodiment, a method treating a patient in need of glucocorticoid therapy, comprising administering to the patient a compound that induces genomic activity mediated by the glucocorticoid receptor without substantially inducing nongenomic activity mediated by the glucocorticoid receptor is described. The effect of the test compound on apoptosis and or cell detachment is then determined. If the compound activates the transcription of the glucocorticoid response element reporter gene construct without substantially effecting apoptosis or cell detachment, assaying the compound for anti-inflammatory activity.

Nongenomic activity can be measured by techniques known in the art. One technique to assess nongenomic activity is to evaluate intracellular calcium levels through stress activated channels. Increased intracellular calcium levels is indicative of non-genomic activity. Levels of intracellular calcium can be monitored using gadolinium as a blocking agent, as is shown in FIG. 8. Alternatively, nongenomic activity can be monitored by assessing JNK activity. Increases in JNK activity is indicative of nongenomic activity. JNK activity can be assessed for example, by monitoring, JUN phosphorylation. In one embodiment, JUN phosphorylation can be determined by antibodies to the phosphorylated JUN in a radioimmunoassay (RIA). Another technique for measuring the JNK activity is to measure Pyk2 activity. Pyk2 activity can be monitored using an RIA with antibodies directed to the phosphorylated pyk2, as is shown is FIG. 4B. Additionally, monitoring cell attachment is an indicator of nongenomic activity. Cell attachment can be assessed by quantification of cytoplasmic projections.

Also described is a method of screening for a compound that induces genomic activity mediated by the glucocorticoid receptor without substantially inducing non-genomic activity mediated by the glucocorticoid receptor, comprising a method of screening for a compound that induces genomic activity mediated by the glucocorticoid receptor without substantially inducing non-genomic activity mediated by the glucocorticoid receptor, comprising contacting a first cell with a test compound wherein the first cell has been transfected with a DNA sequence encoding a glucocorticoid receptor or functional variant of a glucocorticoid receptor and a glucocorticoid response element-reporter gene construct. The effect of the test compound on the transcription of the glucocorticoid response element reporter gene construct is then determined. A second cell is contacted with the test compound and the effect of the test compound on apoptosis and/or cell detachment of the second cell is determined. If the compound activates the transcription of the first cell without substantially effecting apoptosis and/or cell detachment of the second cell, assaying the compound for anti-inflammatory activity.

Also contemplated are compounds and glucocorticoid compounds identified by the-methods described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A and 1B are bar graphs showing the % of apoptotic cells in the presence of the glucocorticoid receptor antagonist, RU486, the protein synthesis inhibitor, cycloheximide or the RNA synthesis inhibitor, actinomyosin D. These graphs demonstrate that glucocorticoid-induced osteocyte apoptosis is mediated by the glucocorticoid receptor, but does not require new gene transcription.

FIG. 2A is a fluorescent micrograph showing the nuclear morphology of a cell undergoing apoptosis. FIG. 2B is a fluorescent micrograph showing the cytoplasmic extensions. FIG. 2C is a graph showing glucocorticoid induced apoptosis over time compared to etoposide induced apoptosis. FIG. 2D is graph showing the effect of dexamethasone compared with etoposide on cell projections. These results illustrate that glucocorticoid-induced osteocyte apoptosis is preceded by changes in cell attachment.

FIG. 3A is a schematic showing an apoptotic cell. FIG. 3B is a series of micrographs showing apoptosis and cell attachment. FIGS. 3C and FIG. 3D graphically show the effect of caspase 3 inhibitor on osteocytes. These results show that glucocorticoid-induced changes in cell attachment are the cause of rather than the result of caspase 3 activation and apoptosis.

FIG. 4A is a schematic showing the differences of the kinase domain of the various kinases or autophosphorylation deficient strains compared to wild type. FIG. 4B is a western blot demonstrating the phosphorylation levels of Pyk2. FIG. 4C graphically demonstrates the effect of overexpression of FAK, pyk2 and two dominant negative pyk2 mutants on glucocorticoid induced apoptosis. These results illustrate that FAK prevents, whereas Pyk2 kinase activity and phosphorylation are required for glucocorticoid-induced apoptosis

FIG. 5A is a schematic showing the effect of the siRNA and recovery. FIG. 5B graphically shows the effect of siRNA with Real time PCR for murine Pyk2 on glucocorticoid-induced apoptosis. FIG. 5C is Western blot analysis of siRNA for murine Pyk2 on glucocorticoid-induced apoptosis. These results illustrate that the expression of murine Pyk2 was silenced with small interference RNAs and recovered by transfection with human Pyk2 fused to GFP, wild type or kinase deficient.

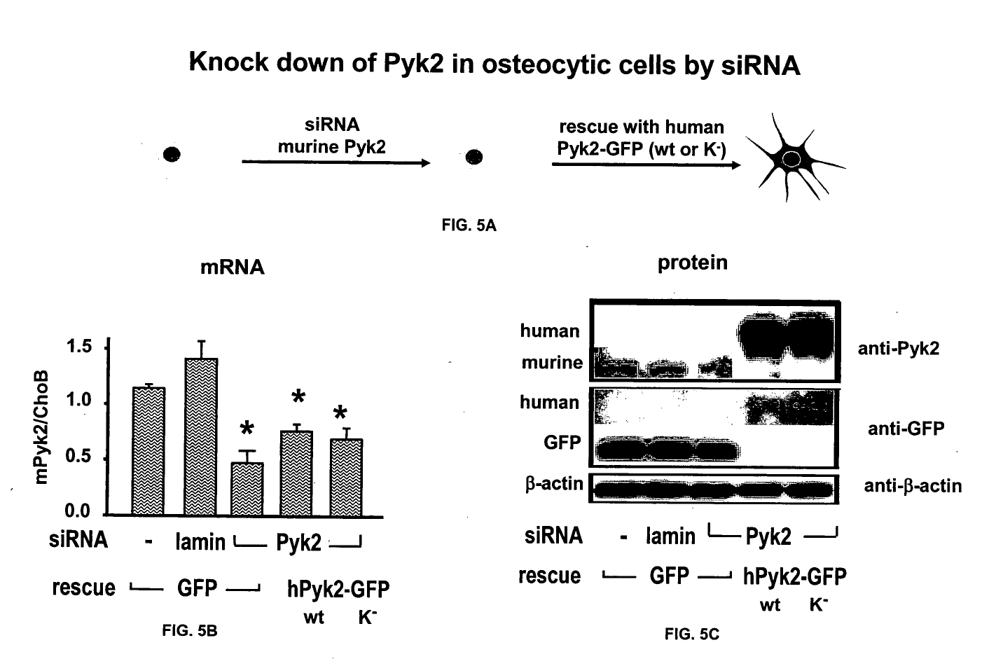

FIG. 6A is a series of micrographs demonstrating pyK2 is required for glucocorticoid induced apoptosis and changes in cell attachment. FIGS. 6B and 6C are bar graphs demonstrating that pyK2 is required for glucocorticoid induced apoptosis and changes in cell attachment.

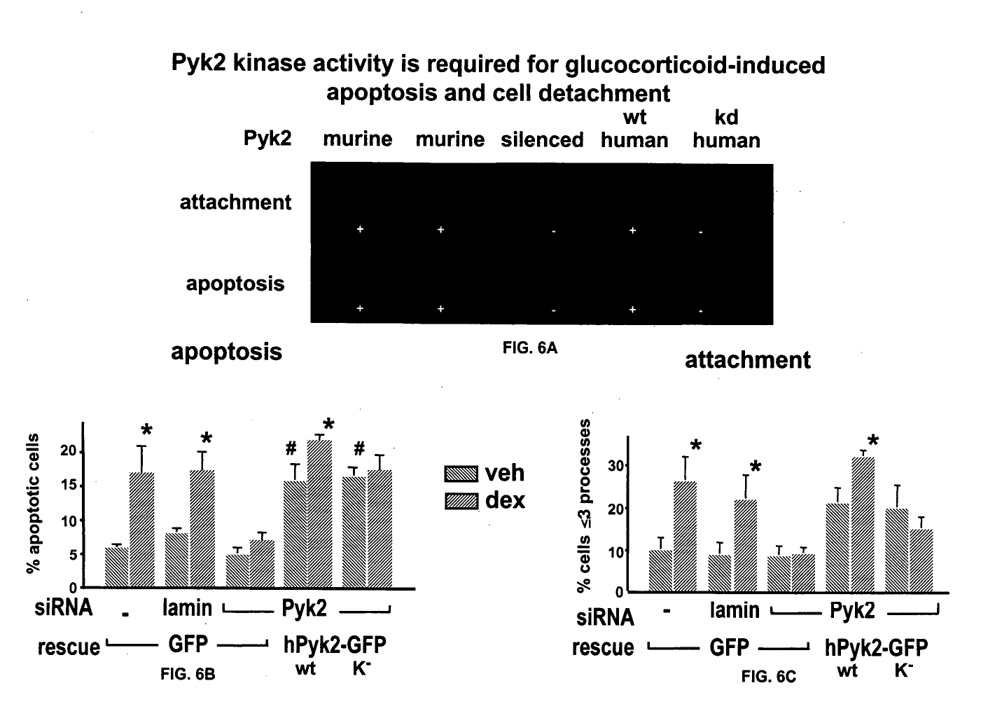

FIG. 7A is a schematic demonstrating the mechanisms involved in glucocorticoid-induced apoptosis. FIG. 7B is a bar graph assaying downstream substrates of Pyk2 using specific pharmacological inhibitors that block several cytoplasmic kinases potentially activated. This demonstrates that inhibition of JNK with SP600125 blocked dexamethasone-induced apoptosis but inhibiting ERK, Src, p38 or PI3K did not have any effect. FIG. 7C shows blockade of JNK activity also prevented dexamethasone-induced changes in cell attachment.

FIG. 8A is a schematic demonstrating the mechanisms involved in calcium entry through stress activated channel for glucocorticoid-induced apoptosis. FIG. 8B is a bar graph demonstrating the effect of different inhibitors on calcium measurement. It demonstrates that calcium entry through stress activated channels is required for glucocorticoid-induced apoptosis.

FIG. 9 is a schematic depicting the proposed model of glucocorticoid induced apoptosis on osteocytes.

FIG. 10 is a schematic depicting that cell survival and attachment are controlled by the focal adhesions.

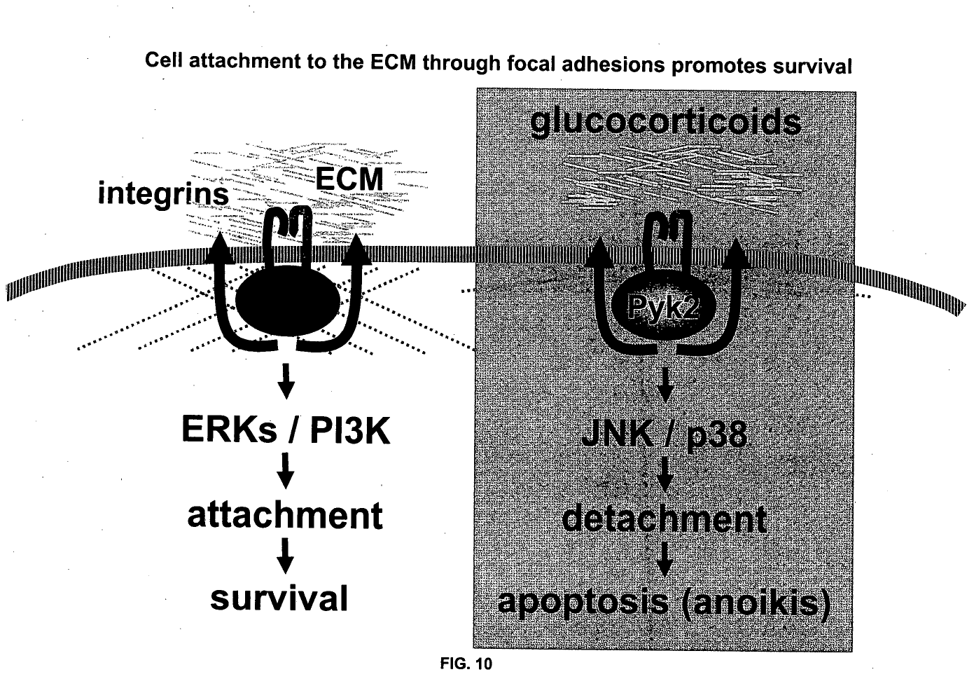

### DETAILED DESCRIPTION OF THE INVENTION

A description of preferred embodiments of the invention follows.

Glucocorticoid-induced bone disease is characterized by decreased bone formation and in situ death of isolated segments of bone (osteonecrosis) suggesting that glucocorticoid excess, a common cause of osteoporosis, may affect the birth or death rate of bone cells thus reducing their numbers.

The invention described herein pertains to the elucidation of the molecular mechanism of the pro-apoptotic effect of glucocorticoids (GC) on osteocytes. In particular, the invention pertains to identifying compounds that are potent anti-inflammatory agents but cause reduced bone loss compared with currently used glucocorticoid compounds. The present invention also provides methods to screen for compounds that retain the anti-inflammatory properties of glucocorticoids yet do not result in the detrimental effect of bone loss.

The findings described herein indicate that GC promote osteocyte apoptosis via a receptor-mediated mechanism that does not require gene transcription and that results from non-genomic, rapid activation of the proapoptotic kinases Pyk2 and JNK, followed by inside-out signaling which favors cell detachment-induced apoptosis or anoikis. In contrast, the immunosuppressing effects of GC are known to be mediated via genomic mechanisms. Therefore, these findings indicate that actions of GC on bone can be mechanistically dissected from their immunosuppressive and/or anti-inflammatory effects. This knowledge provides new tools to find glucocorticoid ligands or compounds that possess genomic activity but lack Pyk2 kinase activation ability (nongenomic activity). Treatment with these compounds will allow one to preserve the beneficial immunosuppressive actions of GC in the absence of deleterious effects on bone.

Mechanism of Action

Glucocorticoids have been shown to induce apoptosis of osteocytes and osteoblasts in vivo and in vitro (Weinstein R. S., et al., J. Clin. Invest., 102(2):274-282(1998); O'Brien, C. A. et al., Endocrinology, 145(4):1835-41(2004) and Weinstein R. S. et al., Endocrinology, 145(4):1980-7 (2004)). Furthermore, specific blockade of glucocorticoid action in cells of the osteoblastic lineage by overexpressing, the glucocorticoid degrading enzyme, 11bHSD2, under the control of the osteocalcin promoter prevents osteocyte apoptosis as well as the loss of strength induced by glucocorticoid administration to mice.

This evidence indicates that apoptosis of osteocytes, by direct actions on these cells, contributes to the bone fragility syndrome that characterizes glucocorticoid-induced osteoporosis. The experiments described herein, elucidate the molecular mechanism of the pro-apoptotic effect of glucocorticoids on osteocytes and utilize this discovery to develop methods of identifying compounds that have the advantageous properties of glucocorticoids without the undesirable bone loss effects.

Specifically, it is shown herein, that glucocorticoid-induced apoptosis is mediated by the glucocorticoid receptor, but it does not require new gene transcription. The glucocorticoid induced apoptosis of osteocytic cells is preceded by changes in cell attachment leading to detachment. New protein or RNA synthesis is not required. The results further indicate that glucocorticoid-induced osteocyte apoptosis is preceded by changes in cell attachment. Both cell survival and attachment are controlled by the focal adhesions, sites at the plasma membrane in which integrins assemble with cytoskeletal and signaling molecules, such as the focal adhesion kinase FAK.

In other cell types, it has been shown that FAK promotes cell attachment and activation of survival kinases, such as ERKs and PI3K. Conversely, the proline-rich tyrosine kinase 2 (Pyk2), which exhibits high homology with FAK, has the opposite effects. Pyk2, a member of the focal adhesion kinase (FAK) family, is a mediator of G-protein-coupled receptors and may be involved in the regulation of the MAP kinase and JNK signal pathways. Results shown herein, indicate that Pyk2 induces reorganization of the cytoskeleton, cell detachment and activation of pro-apoptotic kinases such as JNK and p38 leading to detachment-induced apoptosis or anoikis.

Early studies suggested that some upstream regulators such as integrins, platelet-derived growth factor (PDGF), stress signals, or interleukin-2 could induce Pyk2 activity by modulating the phosphorylation of Pyk2. Changes in the composition and activity of molecules in the focal adhesions results in changes in the interaction between cells and their extracellular matrix. This phenomenon is referred to as inside-out signaling mediated by integrins.

Various constructs were made to investigate the involvement of Pyk2. The effect of glucocorticoids in cells overexpressing wild type FAK or Pyk2, a dn Pyk2 mutant that lacks kinase activity, called K-, or a dn Pyk2 mutant that cannot be autophosphorylated in tyrosine 402, called Y402F, were investigated by evaluating nuclear morphology of cells co-transfected with nGFP.

As described herein, cells transfected with the vector alone exhibited apoptosis in response to dexamethasone or etoposide, cells overexpressing FAK were protected from dexamethasone-induced apoptosis, but not from etoposide-induced apoptosis. This indicates that that dexamethasone and etoposide induce apoptosis by different mechanisms. Furthermore, cells overexpressing Pyk2 exhibited higher level of basal apoptosis, but the cells remained responsive to both dexamethasone or etoposide. Increased basal apoptosis was also observed in cells transfected with the kinase deficient Pyk2, but not with the autophosphorylation deficient Pyk2. However, cells overexpressing either Pyk2 mutant lost the proapoptotic response to dexamethasone, but not to etoposide. Similarly, knocking down pyk2 expression abolished the glucocorticoid induced apoptosis and cell attachment. Transfection with wild type pyk2, but not with kinase deficient pyk2, rescued the response to dexamethasone.

This evidence demonstrates that glucocorticoids induce not only apoptosis but also changes in osteocyte attachment. Glucocorticoids induce osteocyte apoptosis by activating Pyk2, which in turn competes with FAK and results in inside-out signaling that favors detachment and apoptosis led to the development of selective assays for identifying glucocorticoids with anti-inflammatory activity without the adverse effects generated by the activation nongenomic activity, in particular, the activation of Pyk2 or other members of the JNK pathway.

Method for Inducing a Glucocorticoid Response

Methods are described herein of identifying anti-inflammatory compounds by dissociating a genomic effect mediated by the glucocorticoid receptor from the undesirable nongenomic effect mediated by the glucocorticoid receptor. In one embodiment, the invention includes a method for selecting a compound that activates genomic effect without substantially inducing a nongenomic effect. This method includes contacting the receptor with a compound that sufficiently interacts with the receptor in a manner that causes the receptor to mediate a genomic effect without inducing a significant or substantial nongenomic effect. It is considered that a compound does not induce a “significant” or “substantial” effect does not produce a 10%, preferably 20%, more preferably, 50%, increase in apoptosis or another indicator of nongenomic activity compared with basal levels.

In another aspect of the invention, a compound (including but not limited to a peptide or protein or a ligand antagonist) can be administered that competes with an endogenous or exogenous compound of the glucocorticoid receptor for a binding site on the receptor as a means to mask the activity of that compound-binding interaction. As one example, a peptide or compound antagonist can be administered that binds with the compound binding domain in a way that only activates the genomic activity and prevents the compound from inducing a nongenomic effect. The compound can inhibit the transcriptional activity of the receptor by interfering with the receptor from forming a protein-protein interaction with the compound binding domain that is responsible for the nongenomic activity. Additionally, the compound can exist as a complex having at least two components such that when the components combine, the complex selectively activates the desirable genomic activity of the receptor but does not activates the nongenomic activity of a glucocorticoid receptor.

GC Receptor

The glucocorticoid receptor is present in glucocorticoid responsive cells, such as osteocytes, where it resides in the cytosol in an inactive state until it is stimulated by an agonist. Upon stimulation the glucocorticoid receptor translocates to the cell nucleus where it specifically interacts with DNA and/or protein(s) and regulates transcription in a glucocorticoid responsive manner. Two examples of proteins that interact with the glucocorticoid receptor are the transcription factors, API and NFκB. Such interactions result in inhibition of API and NFκB-mediated transcription and are believed to be responsible for some of the anti-inflammatory activity of endogenously administered glucocorticoids. In addition, glucocorticoids may also exert physiologic effects independent of nuclear transcription. Biologically relevant glucocorticoid receptor agonists include cortisol and corticosterone. Many synthetic glucocorticoid receptor agonists exist including dexamethasone, prednisone and prednisilone. RU486 is an example of a glucocorticoid receptor antagonist. By definition, glucocorticoid receptor antagonists bind to the receptor and prevent glucocorticoid receptor agonists from binding and eliciting glucocorticoid receptor mediated events, including transcription.

The glucocorticoid receptor (GR) has an essential role in regulating human physiology and immune response. Glucocorticoids which interact with GR have been shown to be potent anti-inflammatory agents. Despite this benefit, glucocorticoidal GR ligands are not selective. Side effects associated with chronic dosing are believed to be the result of cross-reactivity with other nuclear receptors such as estrogen, progesterone, androgen, and mineralocorticoid receptors which have somewhat homologous ligand binding domains.

As used herein, “a functional variant of a glucocorticoid receptor” varies structurally, from the wild type receptor, but still retains the ability to affect the genomic and non-transcriptional pathway. Permissible structural variations include variations in amino acid sequence or length of the protein. A variant can be a synthetic or artificial receptor, that maintains the activity of the glucocorticoid receptor but is slightly different.

The term “mediated by the glucocorticord receptor” means any activity that results from interaction (e.g., binding) of a compound with the glucocorticoid receptor for example, biological and physiological effects caused by activation of glucocorticoid receptors including transcriptional activity and downstream effects resulting from the transcriptional activity.

“Genomic activity mediated by the glucocorticoid receptor” means transcriptional activity mediated by GRE in the target genes or by interaction of the glucocorticoid receptor with transcription factors (e.g., NFκB and AP-1) and the target genes.

“Nongenomic activity mediated by the glucocorticoid receptor” means activity other than genomic activity resulting from activation of glucocorticoid receptor.

Selective GR repressors, agonists, partial agonists and antagonists of the present disclosure can be used to influence the basic, life-sustaining systems of the body, including carbohydrate, protein and lipid metabolism, and the functions of the cardiovascular, kidney, central nervous, immune, skeletal muscle, and other organ and tissue systems. The term “mediated by” the glucocorticoid receptor includes any activities that result form an interaction (for example, binding) of a compound with the receptor.

In this regard, GR modulators have proven useful in the treatment of inflammation, tissue rejection, auto-immunity, various malignancies, such as leukemias and lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia, and Little's syndrome.

GR modulators are especially useful in disease states involving systemic inflammation such as inflammatory bowel disease, systemic lupus erythematosus, polyartitis nodosa, Wegener's granulomatosis, giant cell arteritis, rheumatoid arthritis, osteoarthritis, hay fever, allergic rhinitis, urticaria, angioneurotic edema, chronic obstructive pulmonary disease, asthma, tendonitis, bursitis Crohn's disease, ulcerative colitis, autoimmune chronic active hepatitis, organ transplantation, hepatitis, and cirrhosis. GR active compounds have also been used as immunostimulants and repressors, and as wound healing and tissue repair agents.

GR modulators have also found use in a variety of topical diseases such as inflammatory scalp alopecia, panniculitis, psoriasis, discoid lupus erythematosus, inflamed cysts, atopic dermatitis, pyoderma gangrenosum, pemphigus vulgaris, bullous pemphigoid, systemic lupus erythematosus, dermatomyositis, herpes gestationis, eosinophilic fasciitis, relapsing polychondritis inflammatory vasculitis, sarcoidosis, Sweet's disease, type 1 reactive leprosy, capillary hemangiomas, contact dermatitis, atopic dermatitis, lichen planus, exfoliative dermatitus, erythema nodosum, acne, hirsutism, toxic epidermal necrolysis, erythema multiform, cutaneous T-cell lymphoma.

Selective antagonists of the glucocorticoid receptor have been unsuccessfully pursued for decades. These agents would potentially find application in several disease states associated with Human Immunodeficiency Virus (HIV), cell apoptosis, and cancer including, but not limited to, Kaposi's sarcoma, immune system activation and modulation, desensitization of inflammatory responses, IL-1 expression, anti-retroviral therapy, natural killer cell development, lymphocytic leukemia, and treatment of retinitis pigmentosa. Cogitive and behavioral processes are also susceptible to glucocorticoid therapy where antagonists would potentially be useful in the treatment of processes such as cognitive performance, memory and learning enhancement, depression, addiction, mood disorders, chronic fatigue syndrome, schizophrenia, stroke, sleep disorders, and anxiety.

Nongenomic Activity Evaluation

Nongenomic activity can be evaluated by various methods, in vivo and in vitro as described herein. Apoptosis can be used to evaluate the nongenomic activity of compounds in the screening methods described herein. Apoptosis can be assessed using techniques known in the art. For example, cells can be transfected with Green Fluorescent Protein localized to the nucleus to assist in assessing changes in nuclear morphology correlating to apoptosis. Apoptosis can also be determined using Terminal Uridine Deoxynucleotidal Transferase Nick End Labeling, In Situ End Labeling, Hoechst staining, or assessing caspase-3 activity.

Additionally, nongentropic activity can be assessed by determining the activation of a signal transduction pathway, for example, the JNK pathway, or a member of the pathway, such as pyk2 (Yu H, et al., J. Biol. Chem, 271(47):29993-29998 (1996) and Tokiwa G. et al., Science, 273(5276):792-794(1996)). Nongenomic (nongenotropic) activity can be evaluated by determining activity of JNK. The nongenomic activity can be determined by 1) changes in intracellular calcium as determined by gadolinium, 2) pyk2 phophorylation by RIA using antibodies to phosphorylated pyk2, 3) JNK activation determining JUN phosphorylation by RIA using antibodies to phosphoylated JUN and 4) by changes in cell attachment by quantifying the number of cytoplasmic projections. Other methods for assessing nongenomic activity are discussed below.

Genomic Activity Evaluation

The degree of genomic activity can be assessed using techniques known in the art. These techniques included in vitro and in vivo testing. In certain embodiments, glucocorticoid receptors contacted with a test compound can be isolated and combined with a nucleic acid sequences corresponding to the glucocorticoid response element. A glucocorticoid response element is a sequence that binds the glucocorticoid receptor and acts as a transcription factor to indicate genomic activity. Lack of binding of the glucocorticoid receptor -compound complex to the GRE sequence indicates that the test compound does not affect genomic activity. One of ordinary skill in the art will appreciate that either the unlabeled glucocorticoid or the unlabeled GRE can be immobilized on a solid phase and a labeled steroid or labeled response element as needed can then be added to the solid phase. Detection of the label on the solid phase in the presence of the test compound indicates that the test compound-receptor complex does not bind nucleic acid and therefore, does not affect genomic activity. Alternatively, the glucocorticoid receptor-test compound complex can be used in binding assays with known nuclear transcription factors. Lack of binding of the glucocorticoid receptor-test compound with nuclear transcription factors indicates that the test compound does not inhibit genomic activity.

In another embodiment, the activation of the DNA binding domain of a glucocorticoid receptor is determined by using a non-endogenous DNA sequence encoding an operable glucocorticoid response element operably linked to a reporter gene. Methods of measuring transcription levels are known in the art and include physical isolation and quantification of mRNA as well as more complex procedures. For example, the production of mRNA can be quantified using real time reverse transcription-polymerase chain reaction (real time RT-PCR). When optimally performed, real time RT-PCR can be used to detect transcripts produced at very low levels and can identify RNAs in minute quantities of starting material. Reagents and real time RT-PCR kits are commercially available. Thus, endogenous mRNA levels can be readily monitored to assess the effect of a compound on transcription levels.

Other methods also include assessing expression levels. The expression of genes can be determined using DNA microarray assays. Briefly, nucleic acid, in the form of long double stranded complimentary DNAs or oligonucleotides, is applied to glass microscope slides, either using robot controlled pins or solid phase chemical synthesis. Tens to hundreds of thousands of unique spots of nucleic acid can be applied to each slide. Radioactively or fluorescently labeled sample (probe) DNA is then applied to the slide and after an appropriate period of time, and several washing steps, the hybridized probe nucleic acid is detected using a microscope or scanner. If the nucleic acid identity of each spot on the slide is known, the corresponding nucleic acid in the sample can be detected by the presence of a signal at that spot's location on the slide. Using this method the expression level of thousands of genes can be simultaneously measured in just a few hours. Other methods for assessing genomic activity are discussed below.

Anti-Inflammatory Activity

Anti-inflammatory activity refers to preventing the induction of cytokines and other events that lead to T cell activation. Several models of inflammation are routinely used in the art, including the adjuvant-induced arthritis model and hindlimb inflammation model that are well known in the art (Knight, B. et al., Clin. Exp. Immunol. 90:459-465 (1992) and Henriques, M. G., et al., Braz. J Med. Biol. Res. 20:243-249 (1987)) can be determined by methods know in the art.

Bone Deteriorating Effects

One deteriorating effect on bone is apoptosis. Apoptosis of bone cells can be assessed by obtaining bone cells from the host after administration of the test compound using TUNEL or Hoechst staining. Additionally, bone histomorphometry can be used for directly and precisely analyzing the effect of test compounds on bone tissue. Histology samples are usually obtained by bone biopsy. The effects of a test compound on the apoptosis of bone cells can also be assessed by determining the effect of the test compound on bone mass or bone strength. Bone mass can be assessed using Dual-Energy X-Ray Absorbtiometry (DEXA) or peripheral quantitative computed tomography (pQCT). DEXA can be used throughout a study to monitor bone mineral desitiy. Bone strength can be measured with biomechanical testing.

For example, bone histomorphometric analysis can be done as described below. The distal femora and lumbar vertebrae are fixed in 4° C. Millonig's phosphate-buffered 10% formalin, pH 7.4, embedded undecalcified in methyl methacrylate and stained. The histomorphometric examination is done with a computer and digitizer tablet (OsteoMetrics Inc. Version 3.00, Atlanta, Ga.) interfaced to a Zeiss Axioscope (Carl Zeiss, Inc., Thomwood, N.Y.) with a drawing tube attachment. All cancellous measurements are two-dimensional, confined to the secondary spongiosa and made at X400 magnification (numerical aperture 0.75). The terminology and units used are those recommended by the Histomorphometry Nomenclature Committee of the American Society for Bone and Mineral Research. The trabecular width and osteoid width were measured directly. Trabecular spacing and number are calculated. Only TRAPase-positive cells are included in the osteoclast perimeter. The rate of bone formation (μm² /μm/d) and turnover (%/d) are then calculated and compared to control.

Another indicator for bone density is serum and urine biochemical measurements. Serum osteocalcin is measured using radioimmunoassay using a goat anti-murine osteocalcin and murine osteocalcin as tracer and standard (Biomedical Technologies, Stoughton, Mass.). Urinary free deoxypyridinoline excretion is determined by a microtiter competitive enzyme immunoassay (Pyrilinks-D, Metra Biosystems, Mountain View, Calif.) and expressed as a ratio to the urinary creatinine.

Glucocorticoid-induced bone disease is characterized by decreased bone formation and in situ death of isolated segments of bone (osteonerosis).

Method for Screening a Compound for its Ability to Selectively Induce a Glucocorticoid Response

Described herein are methods for screening compounds that are capable of inducing a genomic effect on the glucocorticoid receptor without substantially inducing a nongenomic effect, and if the compound has the desired activities, further assaying for anti-inflammatory activity. The method can be carried out in a number of ways, both in vivo and in vitro including:

(1) assessing the ability of the compound to appropriately bind to the glucocorticoid receptor without substantially activating nongenomic activities; and then

(2) measuring the biological activity of the test compound to assess its ability to induce a target genomic effect without substantially inducing a non genomic effect.

Steps 1 and 2 can be combined by simply assaying for compounds that selectively induce genomic effect without substantially inducing the nongenomic effect in an in vitro cell-based assay.

An additional embodiment of the present invention is a method of making a tissue culture screening system comprising the steps of growing a tissue culture cell line until cell growth has reached appropriate confluence, typically approximately 50% confluence; transfecting the tissue culture with an appropriate expression plasmid of a glucocorticoid receptor and an expression plasmid for a target gene responsive to the receptor; contacting the transfected cells with a test compound; and then determining the effect of the test compound on the expression of the target gene to determine whether the compound induces a genomic effect. In an aspect of this embodiment, it is also assessed whether the compound has induced a undesired nongenomic response. In the screening methods, described herein, a tissue culture cell line can be devoid of the receptor whose biological activity is being tested. In this case the missing receptor is added to the cell line by transfection of a plasmid carrying the cDNA for the missing receptor or a chimera for the deficient receptor. The GRE can also be added via transfection. The receptor responsive enhancer DNA element is selected from the responsive elements corresponding to the specific receptor used in the system. The basal promoter consists of a minimal promoter sequence that contains a “TATA” element capable of binding RNA polymerase II and is selected from any of the commonly used elements. For example, thymidine kinase promoter, ovalbumin promoter and MMTV LTR. The term “reporter gene” as used herein refers to any of the variety of genes which produce a protein when transcription is activated by a test compound or chemical signal. This protein is measured to determine the effect on transcription. Reporter genes are operably linked to a promoter under the control of a response element such as the glucocorticoid response element or the like.

The reporter gene can be selected from any of the variety of genes in which the protein product is easily assayed or detected. One skilled in the art readily recognizes that the protein can be easily assayed by calorimetric, fluorescent; immunochemical, chemical or radiochemical methods. Further, the reporter gene can be a chimera. Examples of various reporter genes which can be used include the bacterial enzyme chloramphenicol acetyl transferase (CAT), luciferase, β-galactosidase, alkaline phosphatase, luciferase, peptide hormones, growth factors and chimeric proteins. In the preferred embodiment genes producing non-native proteins are used. For example, CAT or luciferase is used in mammalian systems because they are not a normally occurring enzymes in mammalian cells and are easy to assay. Thus, the receptor dependent activation of CAT or luciferase in tissue culture cells can be used to identify compounds which directly or indirectly interact with the receptor to activate transcription of a gene.

In another embodiment, a method for screening compounds useful in the treatment of glucocorticoid receptor related diseases or disorders is provided comprising: (i) contacting a cell expressing a natural or artificial glucocorticoid receptor with a test compound; (ii) assessing whether the compound activates a genomic activity mediated by the glucocorticoid steroid receptor by determining the level of transcription induced by the test compound; (iii) assessing whether the compound activated a nongenomic activity mediated by the receptor including but not limited to an intracellular second messenger system, signal transduction pathway, protein kinase signal transduction pathway, Pyk2, JNK signal pathway, regulation of intracellular calcium concentration; secretion; changes in cellular morphology; cell motility; cytoskeletal rearrangements; or apoptosis; and (iv) if the compound or compounds that activate the genomic activity mediated by the receptor without substantially activating the nongenomic activity mediated by steroid receptor, assaying the compound or compounds for anti-inflammatory activity.

In another embodiment, the activation of the DNA binding domain of a glucocorticoid receptor is determined by using a non-endogenous DNA sequence encoding an operable GRE operably linked to a reporter gene. Methods of measuring transcription levels are known in the art and include physical isolation and quantification of MRNA as well as more complex procedures. For example, the production of mRNA can be quantified using real time reverse transcription-polymerase chain reaction (RT-PCR). When optimally performed, real time RT-PCR can be used to detect transcripts produced at very low levels and can identify RNAs in minute quantities of starting material. Reagents and real time RT-PCR kits are commercially available. Thus, endogenous MRNA levels can be readily monitored to assess the effect of a compound on transcription levels. In other embodiments of the present invention, compounds are not screened by assaying changes in the appearance or physical characteristics of a compound in response to a test compound interacting with the receptor.

Alternatively, the expression of genes can be determined using DNA microarray assays. Briefly, nucleic acid, in the form of long double stranded complimentary DNAs or oligonucleotides, is applied to glass microscope slides, either using robot controlled pins or solid phase chemical synthesis. Tens to hundreds of thousands of unique spots of nucleic acid can be applied to each slide. Radioactively or fluorescently labeled sample (probe) DNA is then applied to the slide and after an appropriate period of time, and several washing steps, the hybridized probe nucleic acid is detected using a microscope or scanner. If the nucleic acid identity of each spot on the slide is known, the corresponding nucleic acid in the sample can be detected by the presence of a signal at that spot's location on the slide. Using this method the expression level of thousands of genes can be simultaneously measured in just a few hours.

In still another embodiment, activators of selective nongenomic signalling can be selected by (i) contacting cells expressing a natural or artificial glucocorticoid receptor(s) with a test compound; (ii) determining the amount of pyk2 phosphorylation activation in the cells contacted with the test compound; (iii) determining the amount of transcription in the cells contacted with the test compound; (iv) selecting the cells contacted with the test compound that exhibit transcription activation and minimal phosphorylated pyk2 and as compared to phosphorylated pyk2 in cells contacted with the control glucocorticoid compound, if the compound exhibits transcriptional activation with minimal pyk2 activation, then the compound is further assayed for anti-infective activity. In certain embodiments, the compounds are identified as having nongenomic activity using a high throughput combination screening. This screening includes the use of reporter genes and transcription factors that allow readily quantization of activity.

A further embodiment of the present invention is an assay for identifying a test compound or chemical signal that activates genomic receptor mediated activity without substantially activating nongenomic mediated receptor activity, comprising the steps of growing a tissue culture screening system in appropriate media, wherein the cell line of the tissue culture screening system contains a glucocorticoid receptor having at least one identified nongentropic activity; adding a test compound or chemical signal to the media and measuring the amount of transcription and the amount of nongentropic activity. One skilled in the art will readily recognize that the media chosen depends on the cell line chosen and can be any of the chemically defined culture media known in the art and suitable for the cell line. The receptors and reporter genes can be added by either transient or stable transfection. If the compound has the desired genomic activity without the undesired nongenomic activity, the compound is then further assessed for anti-inflammatory activity.

In another embodiment, a method for screening for a compound is provided comprising the steps of: (i) culturing cells in cell culture media wherein the cells naturally or artificially express a glucocorticoid receptor; (ii) contacting the cells with a test compound; (iii) determining whether the test compound activates nongenomic activity mediated the receptor; (iv) determining whether the test compound activates genomic activity mediated by receptor; (v) if the compound that activates genomic activity but does not substantially activate nongenomic activity of the receptor, assaying the compound for anti-inflammatory activity.

Additional embodiments of the present invention include screening methods using multiple transfectants such that cells can be optionally transfected with glucocorticoid receptors, inducible reporter gene plasmids for detecting genomic activity; or inducible reporter gene plasmids for detecting nongentropic activity. The glucocorticoid receptor is transcriptionally active such that it can induce the expression of one or more reporter genes. Compounds can be screened for desired effects on genomic or nongentropic activity by monitoring the change in reporter gene expression when transfected cells are treated with a test compound. For example, a first reporter gene construct can have a GRE upstream of a reporter gene such that the transfected glucocorticoid receptor encodes a protein that interacts with the GRE to induce the expression of the reporter gene. The cells can be single, double, or triple transfectants, or any other multiple of transfectants.

Those of ordinary skill in the art will recognize that the invention can be practiced using multiple cell lines rather than a single cell line containing multiple transfectants. In one embodiment, a method of screening for anti-inflammatory compounds to treat glucocorticoid receptor related disorders is provided comprising; providing a first cell line transfected with a transcriptionally active glucocorticoid receptor and first reporter-gene construct transcriptionally responsive to the glucocorticoid receptor; providing a second cell line transfected with the transcriptionally active glucocorticoid receptor and a second reporter gene construct wherein transcription of the second reporter gene construct indicates the activation of nongentropic activity, preferably a signal transduction pathway; determining the effect of a test compound on the transcription of the first and second reporter gene constructs; and selecting the compound that induces the activation of the first reporter gene construct without substantially activating the transcription of the second reporter gene construct.

In yet another embodiment of the present invention, a method of screening for compounds effective for the reduction of the adverse bone effects seen with treatment of glucocorticoid receptor related diseases or disorders are described, comprising; administering a test compound to a patient, preferably a mammal, determining the effect of the compound on transcription of genes regulated by the glucocorticoid receptor, determining the effect of the compound on the target nongenomic action; and selecting the compound that does not substantially mediate the nongenomic effect and does substantially activate the transcription of glucocorticoid regulated genes.

Those of ordinary skill in the art will appreciate that the screening methods of the present invention can also be utilized to identify anti-inflammatory compounds that activate genomic receptor activity without activating nongenomic activity. Thus, these compounds can be used to treat glucocorticoid responsive diseases and disorders where genomic activity of the glucocorticoid, for example anti-inflammatory is desired but the nongenomic activity, such as glucocorticoid osteoporosis is not. Thus, in one embodiment, a method for screening compounds for the treatment of glucocorticoid receptor related diseases or disorders is provided comprising: (i) contacting a cell expressing a natural or artificial glucocorticoid receptor, with a test compound; (ii) assessing whether the compound activates a nongenomic activity of the glucocorticoid receptor; (iii) determining the level of transcription induced by the test compound; and (iv) selecting the compound or compounds that activate the genomic activity of the steroid receptor without substantially activating the nongenomic activity of the steroid receptor. Substantially activating the nongenomic activity of a steroid receptor means activating a second messenger system such that the second messenger system induces a nongenomic biological response in the cell. Exemplary biological responses are chemical cascades, including but not limited to those caused by phosphorylation, changes in morphology, secretion, proliferation, DNA synthesis, protein synthesis, and cytoskeletal rearrangements.

Coadministration

In one aspect of the invention, the selected compounds described herein can be administered to a patient to treat a glucocordicoid related diseases or disorders in combination with a second pharmaceutical agent.

When the methods of the invention include coadministration, coadministration refers to administration of a first amount of a selected compound having anti-inflammatory activity or a pharmaceutically acceptable salt, hydrate or solvate thereof and a second amount of at least one compound.

In one aspect of the invention, the second compound can be a compound that reduces the undesirable nongenomic activity, in particular on osteocytes. For example, an inhibitor of stress activated calcium channels, a Pyk2 inhibitor, agonist for FAK, siRNA directed for Pyk2 or a JNK inhibitor.

In an embodiment utilizing RNAi of the invention, small double-stranded interfering RNA (RNA interference (RNAi)) can be used to reduce the effects of the nongenomic activity, thereby, inhibiting apoptosis and thus reduce bone loss. RNAi is a post-transcription process, in which double-stranded RNA is introduced, and sequence-specific gene silencing results, though catalytic degradation of the targeted mRNA. See e.g., Elbashir, S. M. et al., Nature 411:494-498 (2001); Lee, N. S., Nature Biotech. 19:500-505 (2002); Lee, S-K. et al., Nature Medicine 8(7):681-686 (2002) and; the entire teachings of these references are incorporated herein by reference.

RNAI is used routinely to investigate gene function in a high throughput fashion or to modulate gene expression in human diseases (Chi et al., PNAS,100 (11):6343-6346 (2003)).

Introduction of long double stranded RNA leads to sequence-specific degradation of homologous gene transcripts. The long double stranded RNA is metabolized to small 21-23 nucleotide siRNA (small interfering RNA). The siRNA then binds to protein complex RISC (RNA-induced silencing complex) with dual function helicase. The helicase has RNAase activity and is able to unwind the RNA. The unwound siRNA allows an antisense strand to bind to a target. This results in sequence dependent degradation of cognate mRNA. Aside from endogenous RNAi, exogenous RNAi, chemically synthesized or recombinantly produced can also be used.

Pharmaceutical Compositions

A compound identified by the methods herein and selected according to the criteria described in detail herein, can be administered in an effective amount to treat any of the conditions described herein, optionally in a pharmaceutically acceptable carrier or diluent.

The active materials can be administered by any appropriate route for systemic, local or topical delivery, for example, orally, parenterally, intravenously, intradermally, subcutaneously, buccal, intranasal, inhalation, vaginal, rectal or topically, in liquid or solid form. Methods of administering the compound of the invention may be by specific dose or by controlled release vehicles.

A preferred mode of administration of the active compound is oral. Oral compositions will generally include an inert diluent or an edible carrier. The active compound can be enclosed in gelatin capsules or compressed into tablets. For the purpose of oral therapeutic administration, the compound can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; and/or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, chewing gum or the like. A syrup may contain, in addition to the active compounds, sucrose as a sweetening agent and certain preservatives, dyes and colorings and flavors.

The compound or a pharmaceutically acceptable derivative or salts thereof can also be mixed with other active materials that do not impair the desired action, or with materials that supplement the desired action. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; chelating agents such as ethylenediaminetetraacetic acid (EDTA); buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In a certain embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions (including liposomes targeted with monoclonal antibodies to surface antigens of specific cells) are also pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and/or cholesterol) in an inorganic solvent that is then evaporated, leaving behind a thin film of dried lipid on the surface of the container. An aqueous solution of the active compound or its monophosphate, diphosphate, and/or triphosphate derivative(s) is then introduced into the container. The container is then swirled by hand to free lipid material from the sides of the container and to disperse lipid aggregates, thereby forming the liposomal suspension.

The dose and dosage regimen will depend upon the nature of the metabolic disease, the characteristics of the particular active compound, e.g., its therapeutic index, the patient, the patient's history and other factors. The schedule will be continued to optimize effectiveness while balanced against negative effects of treatment. See Remington's Pharmaceutical Science, 17th Ed. (1990) Mark Publishing Co., Easton, Pa.; and Goodman and Gilman's: The Pharmacological Basis of Therapeutics 8th Ed (1990) Pergamon Press. Dose ranges contemplated are about 10 pg/ml to about 10 mg/ml.

For parenteral administration, the active compound will most typically be formulated in a unit dosage injectable form (solution, suspension, emulsion) in association with a pharmaceutically acceptable parenteral vehicle. Such vehicles are preferably non-toxic and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives.

The concentration of the compound in the drug composition will depend on absorption, inactivation, and excretion rates of the drug as well as other factors known to those of skill in the art. It is to be noted that dosage values will also vary with the severity of the condition to be alleviated. Additionally, the active ingredient may be administered at once, or may be divided into a number of smaller doses to be administered at varying intervals of time. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed composition.

The present invention is now illustrated by the following Exemplification, which is not intended to be limiting in any way. All references cited herein are incorporated by reference in their entirety.

Exemplification

Materials and Methods as Prepared for the Examples

Cell Culture and Treatments

The murine long bone-derived osteocytic cell line MLO-Y4 was kindly provided by Dr. Lynda Bonewald (University of Missouri-Kansas City, Kansas City, Mo.). Cells were cultured in phenol red-free AMEM supplemented with 2.5% FBS, 2.5% bovine calf serum, 100 U/ml penicillin and 100 μg/ml streptomycin. Cells were plated at 1-2×10⁴ cells/cm² on collagen type I (Sigma Chemical Co., St. Louis, Mo.) coated plates, as described previously (Kato,Y. et al., J. Bone Min.Res., 12:2014-2023 (1997)).

MLO-Y4 cells stably expressing nuclear green fluorescent protein (nGFP) were obtained using the retroviral vector containing the nGFP, provided by Dr. Charles O'Brien (University of Arkansas for Medical Sciences, Little Rock, Ark.). The SV40 large T antigen nuclear localization sequence (Kalderon,D., et al., Cell, 39:499-509 (1984)) was attached to the carboxy-terminus of the cDNA construct encoding nGFP and subcloned into the pLXSN retroviral vector (Clontech, Palo Alto, Calif.). The plasmid harboring the retroviral construct was transiently transfected into the packaging cell line Phoenix Eco (Grignani, F. et al., Cancer Res. 58:14-19 (1998)) using SuperFect (Qiagen, Santa Clarita, Calif.). Supernatants containing retroviral particles were collected 24-48 h after transfection, and used immediately to infect cell cultures. Subconfluent MLO-Y4 osteocytic cells were exposed to viral supernatants in the presence of 8 μg/ml polybrene for 8 h and then incubated in fresh culture medium for 16 hours. Subsequently, cells were exposed to the supernatants containing the viral particles once more before being trypsinized and plated at low density. Transduced cells were selected by culturing them in the presence of 400 μg/ml of geneticin (G418, Sigma Chemical Co., St. Louis, Mo.) for three weeks (Plotkin,L. I., et al., J. Clin. Invest. 104:1363-1374 (1999)).

Apoptosis was induced in semiconfluent cultures by treatment for the indicated times with the glucocorticoid, dexamethasone (10⁻⁶ M, Sigma Chemical Co., St. Louis, Mo.) or with etoposide (50 μM, Sigma Chemical Co., St. Louis, Mo.). Inhibitors were added 30 minute before the addition of the pro-apoptotic agent at the following concentrations: the RNA synthesis inhibitor actinomycin D (2×10⁻⁶M, Sigma Chemical Co., St. Louis, Mo.), the protein synthesis inhibitor cycloheximide (10⁻⁶ M, Sigma Chemical Co., St. Louis, Mo.), the MEK inhibitor PD98059 (50 μM, New England Biolabs, Beverly, Mass.), the Src inhibitor PP1 (5 μM, BioSource International, Camarillo, Calif.), the p38 inhibitor SB203580 (10⁻⁴ M, Sigma Chemical Co., St. Louis, Mo.), the P13K inhibitor wortmannin (30 nM, Sigma Chemical Co., St. Louis, Mo.), the JNK inhibitor SP600125 (3 μM, EMD Biosciences, Inc, San Diego, Calif.) or the caspase-3 inhibitor Asp-Glu-Val-Asp-aldehide (DEVD-CHO) (50 nM, Biomol Research Lab., Inc., Plymouth Meeting, Pa.).

Following the treatment with the pro-apoptotic agents, cells were fixed for 8 minutes with neutral buffer formalin and stored at 4° C. until analyzed.

Transient Transfections

Cells were transiently transfected using Lipofectamine Plus (Gibco BRL, Gaithersburg, Md.) as previously described (Plotkin,L. I. et. al., J. Biol.Chem. 277:8648-8657 (2002) and Kousteni, S. et al., Cell, 104:719-730 (2001)). Briefly, cells were cultured in the absence of serum or antibiotics for 3 hours in the presence of 20 μl/ml Plus reagent and 10 μl/ml Lipofectamine together with 1.3 mg/ml of each of the following DNA constructs: pCDNA3 vector (obtained from Clontech, Palo Alto, Calif.), wild type FAK (provided by Dr. Sandro Aruffo, Bristol-Myers Squibb Pharmaceutical Research Institute, Princeton, N.J. (Chan,P. Y. et al., Methods Mol Biol. 2000.;99.(1.):109.-25. 269:20567-20574 (1994)), or wild type Pyk2, kinase deficient Pyk2 mutant or autophosphorylation deficient Pyk2 mutant (provided by Dr. Wen-Cheng Xiong, University of Alabama at Birmingham, Birmingham, Ala. (Xiong,W. and Parsons,J. T., J Cell Biol 139:529-53(1997)). All the constructs used in this study have been shown to produce functional proteins. Forty hours after transfection, cells were treated with 10⁻⁶ M dexamethasone or 50 μM etoposide for 6 hours. Subsequently cells were fixed cells were fixed for 8 min with neutral buffer formalin and stored at 4° C. until analyzed.

siRNA

The expression of murine Pyk2 or the irrelevant protein lamin A/C was silenced by treating MLO-Y4 cells 40 nM of the corresponding siRNA for 3 hours. Control cultures were left untransfected. Forty hours after silencing cultures were divided and the expression of Pyk2 was recovered by transfection with human Pyk2 fused to GFP (Pyk2-GFP, provided by Dr. David Sancho, Universidad Autónoma de Madrid, Madrid, Spain (Sancho,D. et al., J Cell Biol., 149:1249-1262 (2000)). As control for the recovery, cells were transfected with GFP (Clontech, Palo Alto, Calif.). Forty hours after transfection cells were treated with 10⁻⁶ M dexamethasone or 50 μM etoposide for 6 hours. Subsequently cells were fixed cells were fixed for 8 min with neutral buffer formalin and stored at 4° C. until analyzed.

The expression of murine or human Pyk2 was determined in parallel cultures, by Western blot analysis. Monolayers were washed with cold 20 mM Tris-HCl (pH 7.5), 150 mM NaCl, 1 mM sodium orthovanadate and lysed in 20 mM Tris-HCl (pH 7.5), 150 mM NaCl, 1 mM EDTA, 10 mM NaF, 1 mM sodium orthovanadate, 5 μg/ml leupeptin, 0.14 U/ml aprotinin, 1 mM phenylmethylsulfonylfluoride, and 1% Triton X-100. Insoluble material was pelleted in a microcentrifuge at 14,000×rpm for 10 min. Protein concentration was measured using a Bio-Rad detergent compatible kit (Hercules, Calif.). Proteins were separated on 10% SDS-polyacrylamide gels and electrotransferred to a PVDF membrane. Immunoblottings were performed using a rabbit anti-Pyk2 antibody that recognizes both the human and the murine protein (Upstate, Charlottesville, Va.). The expression of human GFP-Pyk2 was confirmed using a rabbit anti-GFP antibody (Santa Cruz Biotechnology, Santa Cruz, Calif.) and the loading was determined using or a mouse monoclonal anti-β actin (Sigma Chemical Co., Saint Louis, Mo.). Subsequently, blots were exposed to anti-rabbit or anti-mouse antibody conjugated with horseradish peroxidase (Santa Cruz Biotechnology, Santa Cruz, Calif.). Blots were developed using a chemiluminiscence substrate (Pierce, Rockford, Ill.) and the intensity of the bands was quantified using the Versadoc Imaging system (BioRad, Hercules, Calif.).

Quantification of Apoptotic Cells

Dexamethasone- or etoposide-induced apoptosis was assessed in MLO-Y4 cells expressing nGFP by enumerating cells exhibiting chromatin condensation and nuclear fragmentation under a fluorescent microscope. Two hundred and fifty-500 cells from fields selected by systematic random sampling were examinedfor each experimental condition (Plotkin,L. I., et al., J. Clin.Invest. 104:1363-1374 (1999)).

Quantification of Cell Detachment

The effect of dexamethasone or etoposide on cell detachment was quantified by enumerating the number of cytoplasmic processes of MLO-Y4 cells expressing GFP. Cells were classified as having 3 or less cytoplasmic processes or more than 3 cytoplasmic processes.

Image Acquisition

Fluorescent images were collected on an inverted microscope (Axiovert 200, Carl Microscopy, Gottingen, Germany) with a LD A-Plan, 32×/0.40 lens and a low Polaroid DMC Ie, Polaroid Corporation, Cambridge, Mass.), using a filter set for acquisition software was Image-Pro Plus (Media Cybernetics, Silver Spring, Md.).

Statistical Analysis

Data were analyzed by one-way analysis of variance (ANOVA), and the Student-Neuman-Keuls method was used to estimate the level of significance of differences between means.

Results

### EXAMPLE 1

### Glucocorticoid-Induced Osteocyte Apoptosis is Mediated by the Glucocorticoid Receptor, but does not Require New Gene Transcription

MLO-Y4 osteocytic cells were used to test the effect of glucorticoids on apoptosis is the presence of a receptor antagonist, apoptotic agonist, and new protein or RNA synthesis inhibitors.

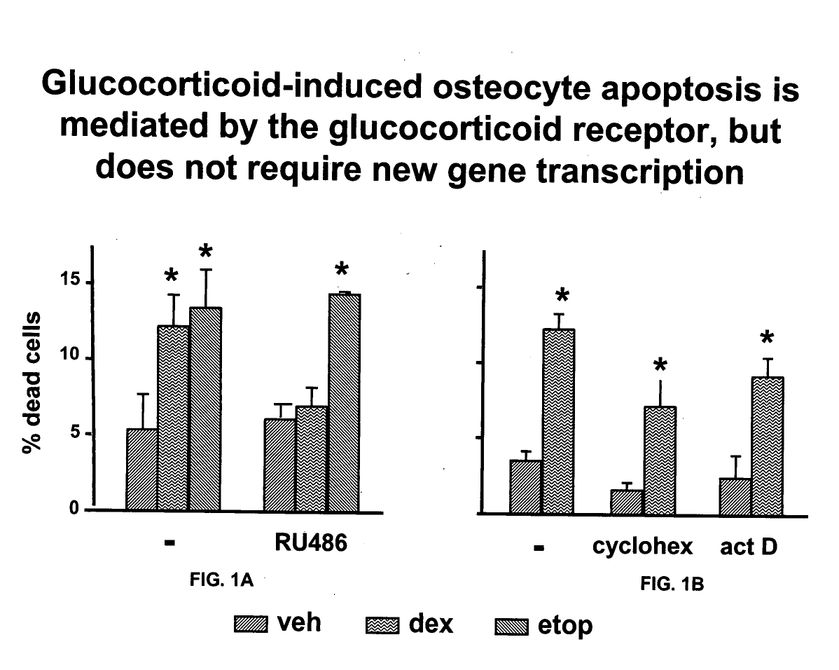

The results of the experiment show (FIG. 1A and FIG. 1B) that apoptosis induced by 6 hour treatment with the glucocorticoid dexamethasone (dex) was prevented by the GC receptor antagonist RU486, whereas etoposide-induced apoptosis was not affected.

In addition, FIG. 1B shows that dexamethasone was able to induce apoptosis in the presence of cycloheximide or actinomycin D, inhibitors of new protein or RNA synthesis.

### EXAMPLE 2

### Glucocorticoid-Induced Osteocyte Apoptosis is Preceded by Changes in Cell-Attachment

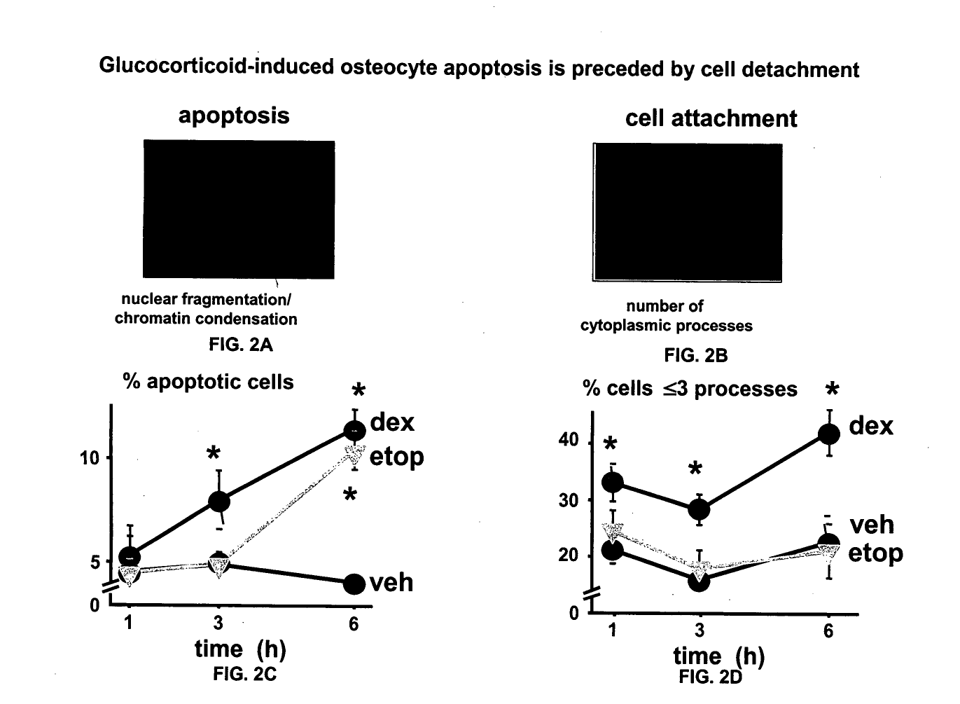

A relationship between glucocorticoid-induced apoptosis and osteocyte attachment is observed using MLO-Y4 cells stably expressing nuclear GFP. Apoptosis and cell attachment were determined in the same cultures by examining nuclear morphology (FIG. 2A) and quantifying the number of cytoplasmic processes (FIG. 2B), respectively. Apoptosis was determined by examining nuclear fragmentation and chromatin condensation and cell detachment by quantifying the number of cytoplasmic processes in the same cells. As shown above, dexamethosone, as well as etoposide, induced apoptosis after 6 hours of treatment. The proapoptotic effect of dexamethasone was already observed at three hours. Strikingly, treatment with dexamethasone increased significantly the number of cells exhibiting three or less cytoplasmic processes, as early as after 1 h of treatment. Although not shown here, this increase was at the expense of a decrease in the number of cells with more than three processes. (FIGS. 2C and 2D). Under basal conditions, the percentage of cells with 3 or less cytoplasmic processes is about 20, the remaining exhibit between four and eight projections.

In contrast, cells treated with etoposide did not exhibit changes in cell attachment during the duration of the experiment. These results indicate that glucocorticoid-induced osteocyte apoptosis is preceded by changes in cell attachment. This demonstrates that glucocorticoid-induced apoptosis preceded by cell detachment is mediated by the glucocorticoid receptor, but it does not require new gene transcription.

### EXAMPLE 3

### Glucocorticoid-Induced Caspase 3 Activation and Apoptosis are Preceded by Changes in Cell Attachment

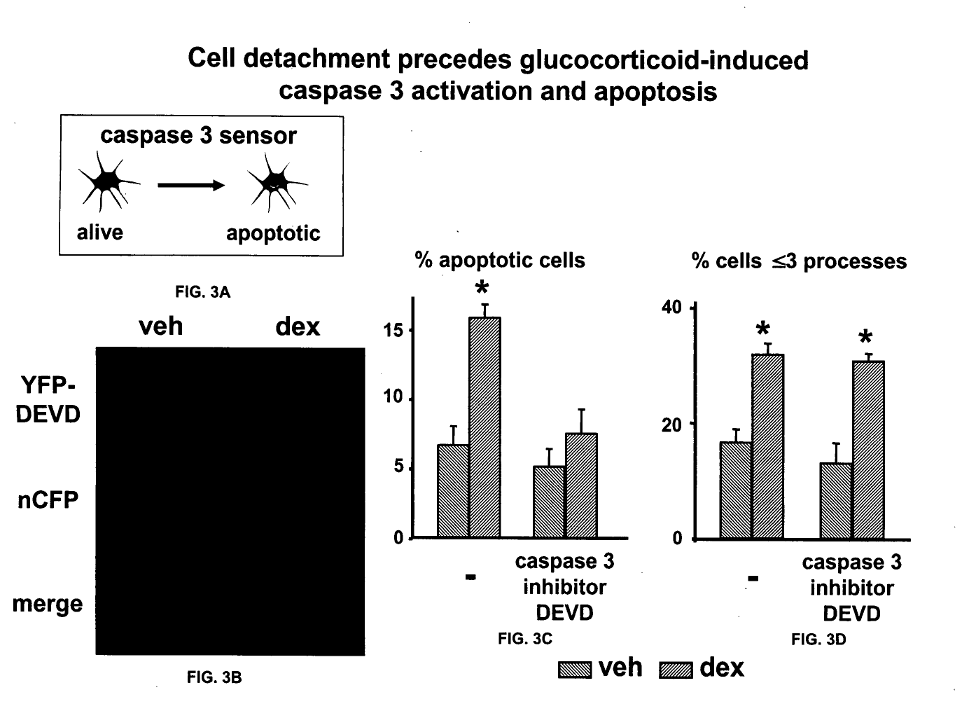

Using a caspase 3 sensor protein, which is located in the cytoplasm of living cells or in the nucleus of apoptotic cells, previous observations were confirmed that dexamethasone increases caspase 3 activity. The numbers in FIGS. 3A-3C are means and standard deviations of the percentage of cells exhibiting nuclear caspase3 upon treatment with vehicle or dexamethasone.

Interestingly, although inhibition of caspase3 activity with DEVD abolished dexamethasone-induced apoptosis, it did not reverse its effect on cell attachment, indicating that changes in cell detachment is the cause of rather that the consequence of caspase 3 activation and apoptosis induced by glucocorticoids.

### EXAMPLE 4

### FAK Prevents, whereas Pyk2 Kinase Activity and Phosphorylation are Required for, Glucocorticoid-Induced Apoptosis

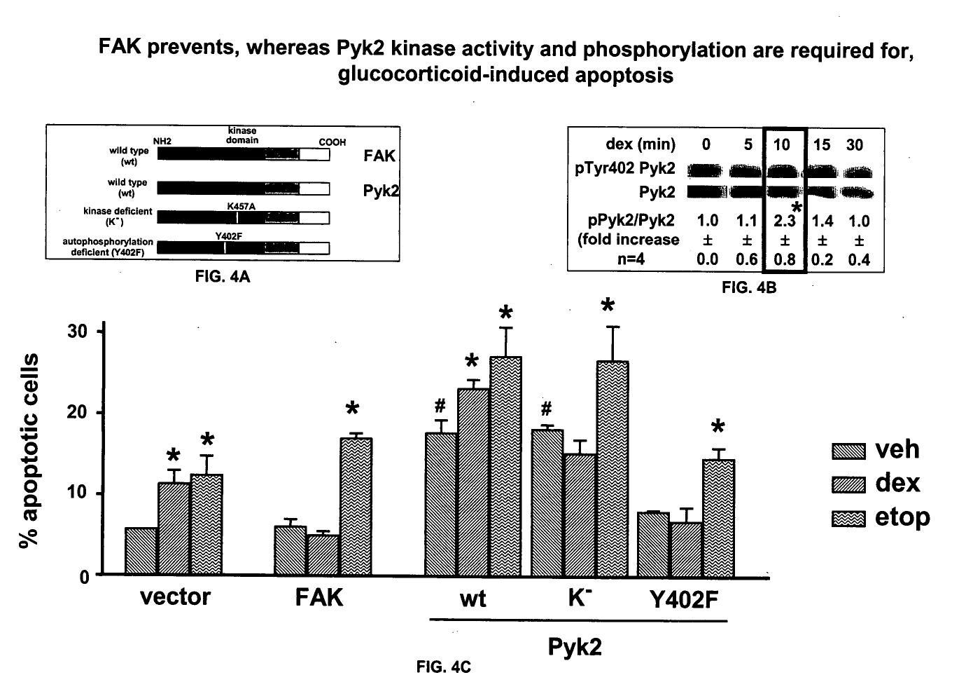

The effect of glucocorticoids in cells overexpressing wild type FAK or Pyk2, a dn Pyk2 mutant that lacks kinase activity, called K-, or a dn Pyk2 mutant that cannot be autophosphorylated in tyrosine 402, called Y402F, were determined by evaluating nuclear morphology of cells co-transfected with nGFP, as shown in FIGS. 4A-4C. The results demonstrate that cells transfected with wild type Pyk2 vector exhibited apoptosis in response to dexamethasone or etoposide, cells overexpressing FAK were protected from dex-, but the cells were not protected from etoposide-, induced apoptosis.

Furthermore, cells overexpressing Pyk2 exhibited higher level of basal apoptosis, but they remained responsive to both dexamethasone or etoposide. Increased basal apoptosis was also observed in cells transfected with the kinase deficient Pyk2, but not with the autophosphorylation deficient Pyk2. However, cells overexpressing either Pyk2 mutant lost the proapoptotic response to dexamethasone, but not to etoposide.

These results demonstrate that the focal adhesion proteins regulate specifically dex-induced apoptosis. Moreover, they show that whereas FAK prevents glucocorticoid-induced apoptosis, Pyk2 kinase activity and phosphorylation are required for this effect. Consistent with the requirement for Pyk2 phosphorylation, dexanmethasone induced rapid phosphorylation of Pyk2 in Y402 that was maximal at 10 min and declined to basal levels by 30 min as determined by Western blotting in four independent experiments.

### EXAMPLE 5

### Knock Down of Pyk2 in Osteocytic Cells by siRNA

The expression of murine Pyk2 was silenced with small interference RNAs and recovered by transfection with human Pyk2 fused to GFP, wild type or kinase deficient. This confirms the requirement of Pyk2 for dexamethasone-induced apoptosis.

Endogenous Pyk2 expression was significantly reduced by siRNA for Pyk2, compared to cells transfected with siRNA for the irrelevant protein lamin A/C or to untransfected cells, shown here by real time RT-PCR for murine Pyk2 or on the right by Western blot analysis.

Furthermore, Pyk2 protein expression was recovered by transfection of either wild type or kinase deficient human Pyk2-GFP, as demonstrated by Western blot analysis using an anti-Pyk2 antibody or an anti-GFP antibody.

### EXAMPLE 6

### Pyk2 is Required for Glucocorticoid-Induced Apoptosis and Changes in Cell Attachment

Consistent with the experiments with transient transfections, knocking down Pyk2 expression abolished not only apoptosis but also the changes in cell attachment induced by dexamethasone. In addition, expression of wild type but not Pyk2 K-restored the responsiveness to dexamethasone. On the other hand, both constructs induced an increase in the basal level of apoptotic cells as well as in the percentage of cells with altered cell detachment. Thus, Pyk2 expression is required for apoptosis as well as for cell detachment induced by glucocorticoids.

### EXAMPLE 7

### Activation of JNK, but not Other Kinases, is Required for Glucocorticoid-Induced Apoptosis and Changes in Osteocyte Attachment

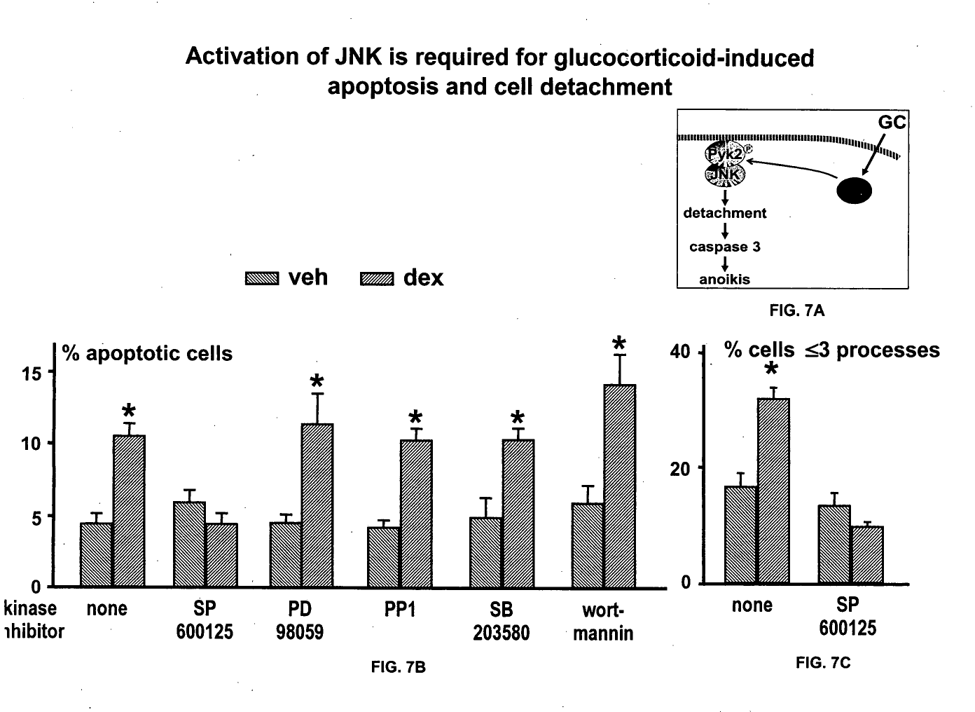

Having established that Pyk2 kinase activity is required for glucocorticoid-induced apoptosis in osteocytic cells, downstream substrates of Pyk2 were then identified using specific pharmacological inhibitors that block several cytoplasmic kinases potentially activated. The inhibition of JNK with SP600125 blocked dexamethasone-induced apoptosis and inhibiting ERK, Src, p38 or PI3K did not have any effect (FIG. 7A-7C).

In addition, blockade of JNK activity also prevented dexamethasone-induced deattachment.

Thus, activation of JNK, but not other kinases, is required for glucocorticoid-induced apoptosis, as well as for changes in osteocyte attachment.

### EXAMPLE 8

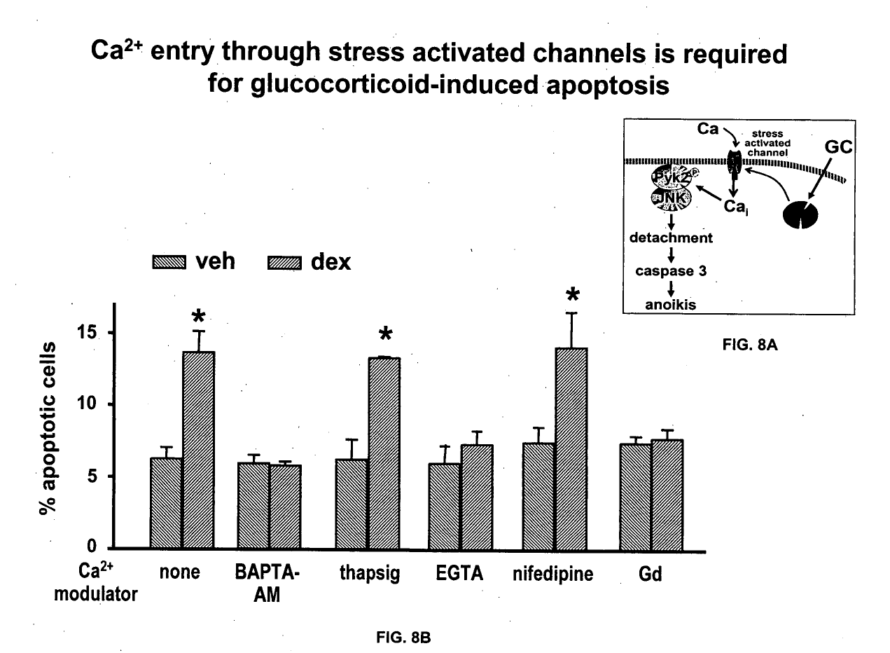

### Calcium Entry through Stress Activated Channels is Required for Glucocorticoid-Induced Apoptosis. See FIG. 8A and 8B.

Extensive evidence indicates that elevation of cytosolic calcium could activate pyk2. Calcium chelation was then investigated to determine whether chelation affected glucocorticoid-induced apoptosis.

BAPTA-AM abolished the effect of glucocorticoids, demonstrating the requirement of changes in intracellular calcium. However, depletion of intracellular stores with thapsigargin had no effect whereas chelation of extracellular calcium with EGTA did. These results indicated that calcium entry from the extracellular medium is required.

It was also found that whereas blockage of L-type voltage-sensitive calcium channels with nifedipine had no effect, inhibition of stress activated channels with gadolinium abolished glucocorticoid-induced anoikis.

### EXAMPLE 9

### Proposed Model

Based on the presented evidence, it is proposed that glucocorticoids promote osteocyte apoptosis via a receptor-mediated mechanism that do not require gene transcription.

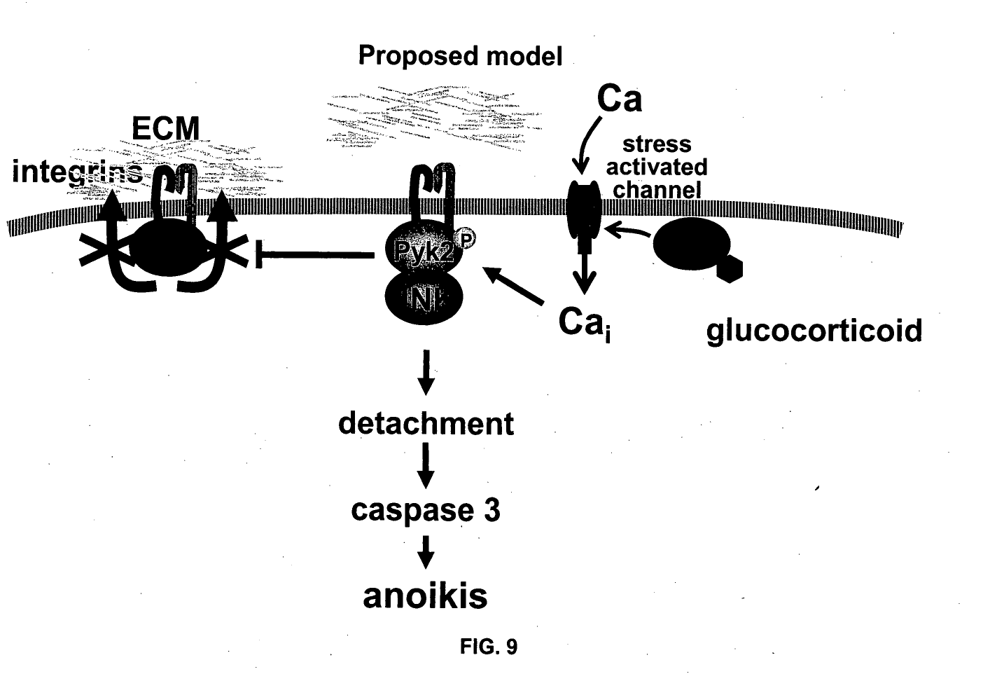

Activation of the glucocorticoid receptor leads to calcium entry through stress activated calcium channels, followed by activation of the focal adhesion related protein Pyk2 and JNK. These kinases interfere with FAK-mediated inside-out survival signaling, leading to cell detachment-induced apoptosis or anoikis (See FIG. 9).

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of screening for an anti-inflammatory compound with reduced bone deteriorating effects, comprising:
a) assessing the ability of the compound to activate genomic activity mediated by a glucocorticoid receptor;
b) assessing the ability of the compound to activate nongenomic activity mediated by the glucocorticoid receptor; and
c) if the compound activates the genomic activity mediated by the receptor without substantially activating the nongenomic activity mediated by the receptor, assaying the compound for anti-inflammatory activity.

2. An anti-inflammatory compound identified by the method of claim 1.

3. A method for screening for an anti-inflammatory compound, comprising:
a) contacting a cell containing a glucocorticoid receptor with a test compound;
b) determining whether the test compound activates the genomic activity mediated by the receptor;
c) determining the level to which the test compound activates the nongenomic activity mediated by the receptor;
d) if the compound activates the genomic activity mediated by the receptor without substantially activating the nongenomic activity of the receptor, assaying the compound for anti-inflammatory activity.

4. The method of claim 3, wherein the nongenomic activity is determined by measuring activity of a member of the JNK pathway compared to a control, wherein increased JNK activity is indicative of activation of nongenomic activity.

5. The method of claim 4, wherein the member is Pyk2.

6. The method of claim 3, wherein nongenomic activity is determined by changes in intracellular calcium though stress activated calcium channels, wherein increase in intracellular calcium is indicative of nongenomic activity.

7. The method of claim 3, wherein the nongenomic activity is determined by measuring cell attachment.

8. The method of claim 3, wherein the glucocorticoid receptor is located on an osteocyte.

9. The method of claim 3, wherein if the compound activates the genomic activity mediated by the receptor and without substantially activating the nongenomic activity by the receptor and has anti-inflammatory activity;
the compound is further assayed to determine the level of bone deteriorating activity.

10. The method of claim 8, wherein if the compound has anti-inflammatory activity and has reduced have bone deteriorating activity; the compound is further assayed in a human for anti-inflammatory activity and bone deterioration activity compared to a control glucocorticoid.

11. A method of treating a patient in need of glucocorticoid therapy, comprising administering to the patient a compound that induces genomic activity mediated by the glucocorticoid receptor without substantially inducing nongenomic activity mediated by the glucocorticoid receptor.

12. The method of claim 1, wherein the genomic activity is determined by a reporter gene operably linked to a glucocorticoid receptor response element.

13. A method of screening for a compound that induces genomic activity mediated by the glucocorticoid receptor without substantially inducing non-genomic activity mediated by the glucocorticoid receptor, comprising:
a) contacting a cell with a test compound wherein the cell has been transfected with:
i) a DNA sequence encoding a glucocorticoid receptor or functional variant of a glucocorticoid receptor;
ii) a glucocorticoid response element-reporter gene construct;
b) determining the effect of the test compound on apoptosis and/or cell detachment; and
c) if the compound activates the transcription of the glucocorticoid response element reporter gene construct without substantially effecting apoptosis or cell detachment, assaying the compound for anti-inflammatory activity.

14. The method of claim 13, wherein if the compound has anti-inflammatory activity, the compound is further assayed to determine the level of bone deteriorating activity.

15. A compound identified by the method of claim 13.

16. A method of screening for a compound that induces genomic activity mediated by the glucocorticoid receptor without substantially inducing non-genomic activity mediated by the glucocorticoid receptor, comprising:
a) contacting a first cell with a test compound wherein the first cell has been transfected with:
i) a DNA sequence encoding a glucocorticoid receptor or functional variant of a glucocorticoid receptor;
ii) a glucocorticoid response element-reporter gene construct; and
determining the effect of the test compound on the transcription of the glucocorticoid response element reporter gene construct;
b) contacting a second cell with the test compound and determining the effect of the test compound on apoptosis and/or cell detachment of the second cell; and
c) if the compound activates the transcription of the first cell without substantially effecting apoptosis and/or cell detachment of the second cell, assaying the compound for anti-inflammatory activity.

17. The method of claim 16, wherein if the compound has anti-inflammatory activity, the compound is further assayed to determine the level of bone deteriorating activity.
